(19) United States
(12) Patent Application Publication
(10) Pub. No.: US 2010/0003712 A1
(43) Pub. Date: Jan. 7, 2010

(54) BIOMARKER FOR ASSESSING RESPONSE TO CHYMASE TREATMENT

(76) Inventors: Zhiming Zhang, Belle Mead, NJ (US); Michael D'Andrea, Cherry Hill, NJ (US); Stanley M. Belkowski, Norristown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/474,413

(22) Filed: May 29, 2009

Related U.S. Application Data
(60) Provisional application No. 61/058,679, filed on Jun. 4, 2008.

(51) Int. Cl. C12Q 1/02; C07K 14/715

(52) U.S. Cl. 435/29; 530/351

(57) ABSTRACT

A biomarker that correlates to treatment with drugs that inhibit chymase is disclosed. This biomarker has been shown to have utility in assessing response to chymase inhibitor compounds. The immunoreactivity or levels of the biomarker is increased upon treatment with chymase inhibitor compounds, thus indicating that this biomarker is involved in chymase activity.

### RELATED APPLICATION

This application is a non-provisional filing of a provisional application, U.S. Ser. No. 61/058,679, filed on Jun. 4, 2008.

### FIELD OF THE INVENTION

The present invention relates generally to the field of pharmacodynamics, and more specifically to materials, methods and procedures to determine drug sensitivity in patients, including in patients with inflammatory or serine protease/chymase mediated diseases. This invention aids in treating diseases and disorders based on patient response at a molecular level.

### BACKGROUND OF THE INVENTION

Human α-chymase, a chymotrypsin-like protease secreted by mast cells, plays an important role in physiological and pathological conditions (for review see Caughey, 2007). Chymase activates several biological mediators such as angiotensin I, big endothelins, interleukin-1β, stem cell factor, and interstitial collagenases, and is involved in the degradation of extracellular matrix. Thus, chymase is believed to play a crucial role in a variety of inflammatory conditions and chymase inhibitors may have the potential for the treatment of certain inflammatory diseases.

A number of drugs that reduce or inhibit the activity of chymase are currently being developed. WO2005073214 provides phosphornic acid and phosphinic acid compounds, including COMPOUND NOs. 1, 2, and 3, as inhibitors of chymase. COMPOUND NO. 1 is a first-in-class, orally active, selective chymase inhibitor (Ki=29 nM). COMPOUND NO. 1 shows little or no cross activity with other serine proteases. The structure of COMPOUND NO. 1 is below.

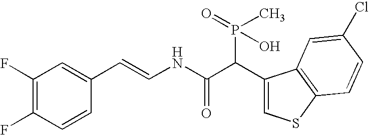

The structure of COMPOUND NO. 2 is:

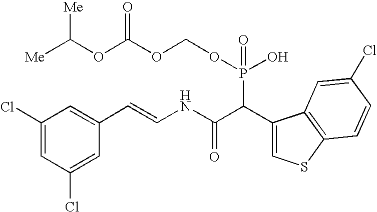

The structure of COMPOUND NO. 3 is

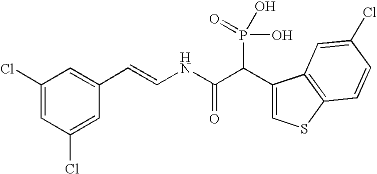

Predictive markers are needed to accurately foretell a patient's response and required dosage to drugs such as COMPOUND NOs. 1, 2, and 3 in the clinic. Such markers would facilitate the individualization of therapy for each patient.

The present invention is directed to the identification of a biomarker that can better predict a patient's sensitivity to treatment or therapy with drugs that reduce or inhibit chymase. The association of a patient's response to drug treatment with this marker can open up new opportunities for drug development in non-responding patients, or distinguish a drug's indication among other treatment choices because of higher confidence in the efficacy. Further, the pre-selection of patients who are likely to respond well to a drug or combination therapy may reduce the number of patients needed in a clinical study or accelerate the time needed to complete a clinical development program (M. Cockett et al., 2000).

A major goal of research is to identify markers that accurately predict a given patient's response to drugs in the clinic; such individualized assessment may greatly facilitate personalized treatment. An approach of this nature is particularly needed in asthma treatment and therapy, where commonly used drugs are ineffective in many patients, and side effects are frequent. The ability to predict drug sensitivity in patients is particularly challenging because drug responses reflect both the properties intrinsic to the target cells and also a host's metabolic properties.

Needed in the art are materials, methods and procedures to determine drug sensitivity in patients in order to treat inflammatory diseases and disorders, particularly chymase mediated diseases, based on patient response at a molecular level. The diseases include, but are not limited to, inflammatory bowel disease, dermal inflammatory disease (e.g. atopic dermatitis), allergic rhinitis, viral rhinitis, asthma, chronic obstructive pulmonary diseases, bronchitis, pulmonary emphysema, acute lung injury (e.g. adult/acute respiratory distress syndrome), psoriasis, arthritis, reperfusion injury, ischemia, hypertension, hypercardia myocardial infarction, heart failure damage associated with myocardial infarction, cardiac hypertrophy, arteriosclerosis, saroidosis, vascular stenosis or restenosis (e.g. associated with vascular injury, angioplasty, vascular stents or vascular grafts), pulmonary fibrosis, kidney fibrosis (e.g. associated with glomerulonephritis), liver fibrosis, post surgical adhesion formation, systemic sclerosis, keloid scars rheumatoid arthritis, bullous pemphigiod and atherosclerosis. Additionally, these compounds can be used for modulating wound healing and remodeling (e.g. cardiac hypertrophy) as well as immune modulation.

The present invention involves the identification of a biomarker that correlates with patient sensitivity to drugs that reduce or inhibit chymase. The presently described identification of marker can be extended to clinical situations in which the marker is used to predict responses to drugs that reduce or inhibit chymase.

IL-18 was originally identified as an IFNγ-inducing factor in the sera and livers of mice treated with Proionibacterium acnes (P. acnes) and lipopolysaccharide (LPS) and believed to play an important role in host defense (Okamura, et al., 1995a, b). Recently, increasing lines of evidence also suggest that IL-18 is a pleiotropic cytokine that enhances both Th1- and Th2-driven immune responses (Nakanishi, et al., 2001). IL-18 is expressed in a variety of cells including Kupffer cells, macrophages, T and B cells, osteoblasts, dendritic cells, and epithelial cells such as keratinocytes and salivary glands (Nakanishi, et al., 2001; Bombardieri, et al., 2004). The precursor protein of human IL-18 (pro-IL-18) is a 24-kDprotein (193 aa) without the usual leader sequence for secretion. The amino acid sequence of human IL-18 is 95% and 65% homologous with that of the macaque and murine IL-18, respectively. The mature form of IL-18 is an 18-kD protein (157 aa) generated by the IL-1β-converting enzyme (ICE, caspase 1), whereas caspase-3 degrades pro-IL-18 into biologically inactive products (Nakanishi, et al., 2001). Recently, Omoto et al. (2006) demonstrated that a recombinant human mast cell chymase was able to cleave a recombinant pro-IL-18 and generate a novel and biologically active fragment of 16 kD. However, this recombinant human mast cell chymase did not cleave the mature form of IL-18.

In the process of investigating a biomarker for chymase inhibitors, it has been determined that a recombinant human chymase cleaves a recombinant mature human IL-18. The digestion by chymase leads to a significant decrease of IL-18 immunoreactivity or levels as determined by commonly used methods such as enzyme linked immunosorbent assay (ELISA). Also chymase specific inhibitors modulate the chymase activity and reverse the decrease of IL-18 immunoreactivity/levels mediated by chymase. Therefore, the reversal of chymase-induced decrease of IL-18 levels may serve as a biomarker for chymase specific inhibitors.

### SUMMARY OF THE INVENTION

The present invention is related to the identification that increased interleukin-18 (IL-18) immunoreactivity or levels is correlated with inhibition of chymase. This marker shows utility in predicting a host's response to a drug and/or drug treatment.

It is an aspect of the invention to provide a method of monitoring the treatment of a patient having a disease treatable by a drug that modulates chymase. This can be accomplished by determining IL-18 immunoreactivity or levels from a patient prior to treatment with a drug that inhibits chymase activity and again following treatment with the drug. Thus, if a patient's response becomes one that is sensitive to treatment by a chymase inhibitor compound, based on a correlation of an observed increase in IL-18 immunoreactivity/levels, the patient's treatment prognosis can be qualified as favorable and treatment can continue. Also, if after treatment with a drug, the patient's IL-18 immunoreactivity/levels does not increase, this can serve as an indicator that the current treatment should be modified, changed, or even discontinued. Such a monitoring process can indicate success or failure of a patient's treatment with a drug, and the monitoring processes can be repeated as necessary or desired.

### DESCRIPTION OF THE FIGURES

FIG. 1 shows Western blot analysis of the cleavage of mature IL-18 by chymase. A. Recombinant mature human IL-18 (rIL-18, 100 ng) was incubated with chymase (w/w 1:25) in PBS at 37° C. for various time points. The digestion products were separated on a 4-12% gradient gel. Western blot analysis was done with a specific monoclonal antibody against human IL-18. Two fragments of ˜16 and ˜12 kDs were detected after chymase digestion (arrows). B. A 100 ng of rIL-18 was incubated with various concentrations of chymase in PBS at 37° C. for 2 h.

FIG. 2 shows the chymase reduced IL-18 immunoreactivity or levels. Four mg/ml of rIL-18 was added to PBS or saliva samples at 37° C. for 1.5 h with or without chymase (80 nM).

FIG. 3 shows the effects of chymase and chymase specific inhibitor COMPOUND No. 3 on IL-18 immunoreactivity/levels in human saliva samples. The saliva samples (1 ml) were incubated with vehicle (DMSO/PBS), chymase (80 nM), or chymase+COMPOUND No. 3 (1 μM) at 37° C. for 3 h.

FIG. 4 shows the effects of chymase inhibitor COMPOUND No. 3 and pan-protease inhibitor on IL-18 levels in human saliva samples. The samples were incubated with vehicle (DMSO/PBS), COMPOUND No. 3 or IX pan protease inhibitors at 37° C. for 1 h.

FIG. 5 shows the effect of chymase on IL-18 levels in human saliva samples. The chymase in 1-300 nM was added to saliva samples and incubated at 37° C. for 1 h.

FIG. 6 shows that COMPOUND NOs. 1, 2, and 3 dose-dependently reversed chymase-induced decrease of IL-18 levels in human saliva.

FIG. 7 shows the effects of various serine proteases and inhibitors on IL-18 levels in saliva samples. The samples were incubated with vehicle, chymase (2 μg/ml), COMPOUND No. 3 (1 μM), chymase+COMPOUND No. 3, chymostatin (CS, 3 μg/ml), chymase+chymostatin, cathepsin (Cat G, 2 μg/ml), Cat G+COMPOUND No. 3, elastase (0.5 μg/ml), elastase+COMPOUND No. 3, proteinase 3 (Prot 3, 2 μg/ml), proteinase 3+COMPOUND No. 3, tryptase (1 μg/ml), or tryptase+COMPOUND 3 at 37° C. for 1 h.

FIG. 8 shows the effects of COMPOUND NO. 1 on the IL-18 levels in human saliva samples after 14 days of dosing in humans.

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

### DEFINITIONS

As used herein, the terms “comprising”, “containing”, “having” and “including” are used in their open, non-limiting sense.

A “biological sample” as used herein refers to a sample containing or consisting of cells or tissue matter, such as cells or biological fluids isolated from a subject. The “subject” can be a mammal, such as a rat, a mouse, a monkey, or a human, that has been the object of treatment, observation or experiment. Examples of biological samples include, for example, sputum, blood, blood cells (e.g., white blood cells), amniotic fluid, plasma, serum, semen, saliva, bone marrow, tissue or fine-needle biopsy samples, urine, peritoneal fluid, pleural fluid, and cell cultures. Biological samples may also include sections of tissues such as frozen sections taken for histological purposes. A test biological sample is the biological sample that has been the object of analysis, monitoring, or observation. A control biological sample can be either a positive or a negative control for the test biological sample. Often, the control biological sample contains the same type of tissues, cells and/or biological fluids of interest as that of the test biological sample. In particular embodiments, the biological sample is a “clinical sample,” which is a sample derived from a human patient.

A “cell” refers to at least one cell or a plurality of cells appropriate for the sensitivity of the detection method. The cell can be present in a cultivated cell culture. The cell can also be present in its natural environment, such as a biological tissue or fluid. Cells suitable for the present invention may be bacterial, but are preferably eukaryotic, and are most preferably mammalian.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by the codons of genes may also be included in a polypeptide.

An “isolated protein” is one that is substantially separated from at least one of the other proteins present in the natural source of the protein, or is substantially free of at least one of the chemical precursors or other chemicals when the protein is chemically synthesized. A protein is “substantially separated from” or “substantially free of” other protein(s) or other chemical(s) in preparations of the protein when there is less than about 30%, 20%, 10%, or 5% (by dry weight) of the other protein(s) or the other chemical(s) (also referred to herein as a “contaminating protein” or a “contaminating chemical”).

Isolated proteins can have several different physical forms. The isolated protein can exist as a full-length nascent or unprocessed polypeptide, or as a partially processed polypeptide or as a combination of processed polypeptides. The full-length nascent polypeptide can be post-translationally modified by specific proteolytic cleavage events that result in the formation of fragments of the full-length nascent polypeptide. A fragment, or physical association of fragments can have the biological activity associated with the full-length polypeptide; however, the degree of biological activity associated with individual fragments can vary.

An isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemical synthesis, as will be apparent to skilled artisans.

The present invention describes the identification that an increase in IL-18 immunoreactivity/levels serves as a useful molecular tool for predicting a response to drugs that affect chymase activity via direct or indirect inhibition or antagonism of the chymase function or activity.

Also provided by the present invention are monitoring assays to monitor the progress of drug treatment involving drugs that interact with or inhibit chymase activity. Such in vitro assays are capable of monitoring the treatment of a patient having a disease treatable by a drug that modulates or interacts with chymase by comparing IL-18 immunoreactivity prior to treatment with a drug that inhibits chymase activity and again following treatment with the drug. Isolated samples from the patient are assayed to determine IL-18 immunoreactivity or levels before and after exposure to a drug, preferably a chymase inhibitor, to determine if a change of the IL-18 immunoreactivity/levels has occurred or not so as to warrant treatment with another drug, or whether current treatment should be discontinued.

In another embodiment, the human chymase inhibitor biomarker can be used for screening therapeutic drugs in a variety of drug screening techniques.

The term “drug” is used herein to refer to a substance that potentially can be used as a medication or in the preparation of a medication. Essentially any chemical compound can be employed as a drug in the assays according to the present invention. Compounds tested can be any small chemical compound, or biological entity (e.g., amino acid chain, protein, sugar, nucleic acid, or lipid). Test compounds are typically small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including, for example, Sigma (St. Louis, Mo.), Aldrich (St. Louis, Mo.), Sigma-Aldrich (St. Louis, Mo.), Fluka Chemika-Biochemica Analytika (Buchs, Switzerland). Also, compounds can be synthesized by methods known in the art.

### EXAMPLES

The Examples herein are meant to exemplify the various aspects of carrying out the invention and are not intended to limit the scope of the invention in any way.

### Example 1

### Cleavage of Recombinant Human Mature IL-18 by Chymase

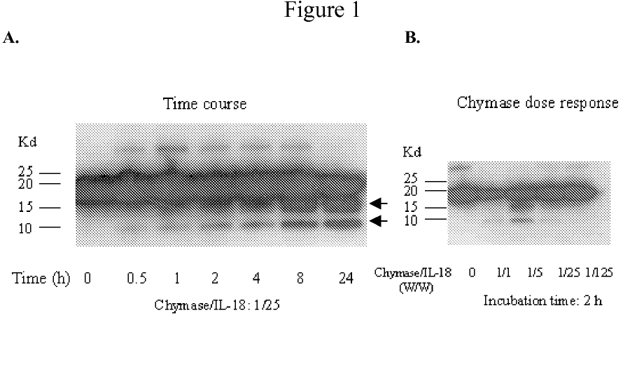

A recombinant human chymase was generated according to Kervinen et al. (2008). A hundred ng of the recombinant mature human IL-18 (rIL-18, R&D Systems, 614 McKinley Place NE, Minneapolis, Minn. 55413) was incubated with the recombinant chymase (w/w 1:25) in PBS at 37° C. for various time points. The digestion products were separated on a 4-12% gradient gel. Western blot analysis was done with a specific monoclonal antibody against human IL-18. As shown in FIG. 1, the recombinant human chymase was able to cleave a mature human IL-18 as two fragments of ˜16 and ˜12 kDs were detected by western blot hybridization analysis. The exact cleavage sites were not clear. Without being bound to any theory, the mature IL-18 may be cleaved by the recombinant human chymase into the ˜16 kD fragment which may be further degraded into the ˜12 kD fragment. Alternatively, the IL-18 may be cleaved by the recombinant human chymase at two distinct sites which resulted in either the ˜16 kD fragment or the ˜12 kD fragment. In addition, the chymase-induced cleavage of rIL-18 was both time (FIG. 1A) and dose dependent (FIG. 1B).

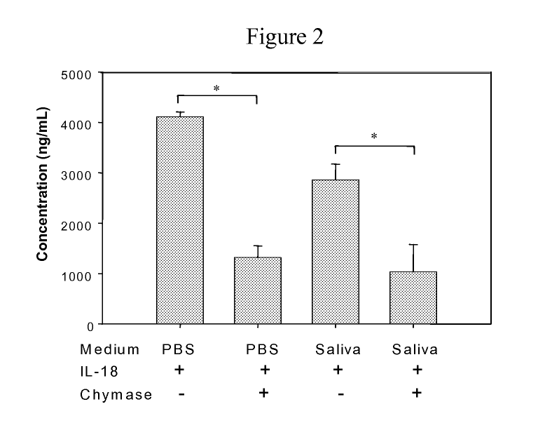

To determine whether the recombinant chymase was able to cleave rIL-18 in conditions similar to that of body fluid, 80 nM of chymase and 4 mg/ml of rIL-18 were added to PBS or saliva at 37° C. for 1.5 h. The digestion was measured using the enzyme-linked immunosorbent assay (ELISA) kit (R&D Systems). The results from ELISA showed, in the presence of the recombinant chymase, the rIL-18 immunoreactivity or levels were significantly decreased from ˜4000 to ˜1300 ng/ml in PBS and from ˜3,000 to ˜1,000 ng/ml in saliva (FIG. 2). Hence, the chymase was able to cleave IL-18 in either PBS or saliva samples and the chymase activities could be detected using ELISA. As the ELISA detection provided quantitative measurement and high-throughput screening capacity, it was used for the following experiments.

### Example 2

### Effects of the Chymase and the Chymase Inhibitors on Endogenous IL-18

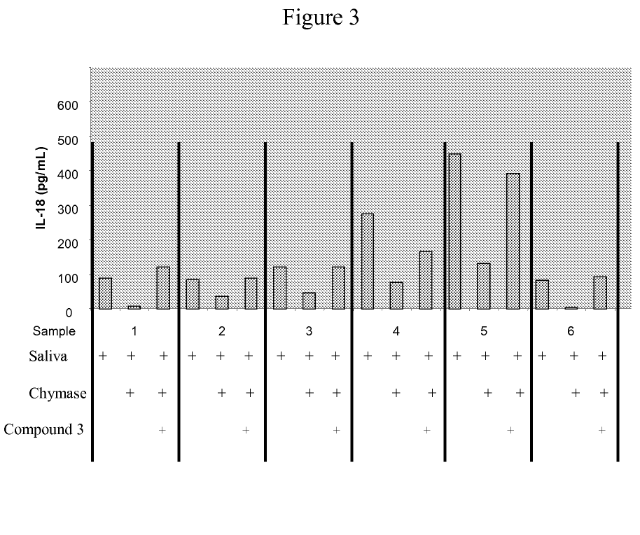

Next, activities of the recombinant chymase and COMPOUND NO. 3 were examined with endogenous IL-18. One ml of saliva samples were collected from six subjects and incubated with vehicle (DMSO/PBS), chymase (80 nM), and chymase+COMPOUND No. 3 (1 μM) at 37° C. for 3 hr. As shown in FIG. 3, endogeous IL-18 was detected in all six subjects with levels ranging from ˜80 to ˜500 pg/ml. The addition of the recombinant chymase resulted in a decrease in the immunoreactivity or levels of endogenous IL-18 in saliva samples. Further, the chymase-induced decrease of IL-18 levels was reversed or prevented by COMPOUND No. 3. In subjects 1-3 and 6, the IL-18 levels were reversed completely whereas in subjects 4 and 5, the IL-18 levels were reverted to a large degree.

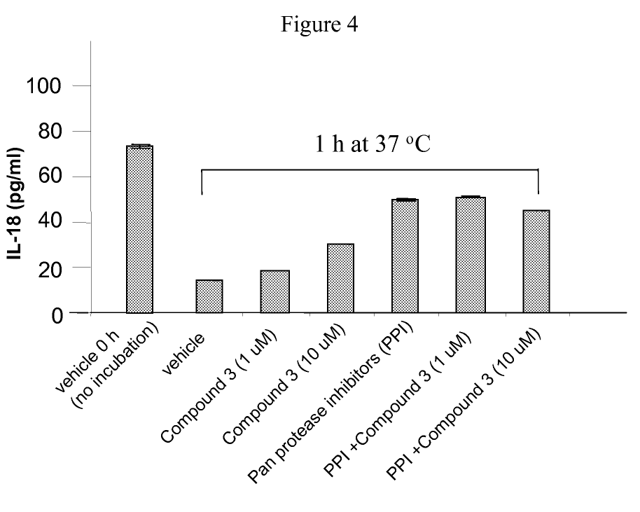

To determine the effects of additional protease inhibitors on the endogenous IL-18 levels, the pan protease inhibitors and/or COMPOUND No. 3 were incubated with saliva samples at 37° C. for 1 hour. The IL-18 levels were detected by ELISA and the results were shown in FIG. 4. Interestingly, the IL-18 levels in samples incubated with the DMSO/PBS vehicle were reduced. COMPOUND No. 3, in either 1 or 10 μM, reversed some of the decrease in the IL-18 levels. Ten μM of COMPOUND NO. 3 increased the IL-18 levels almost two fold compared with 1 μM of COMPOUND No. 3. Further, the pan protease inhibitors were also able to reverse the IL-18 levels. This result suggests that endogenous chymase activity and other proteases activity in saliva samples could reduce IL-18 levels.

### Example 3

### Dose Response Studies of the Chymase and the Chymase Inhibitors

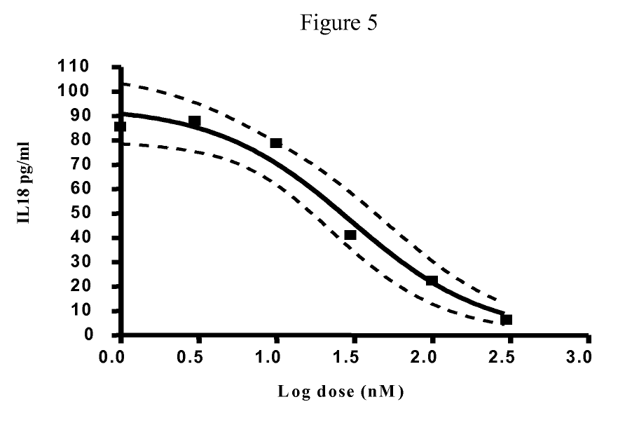

To determine the dose effects of the recombinant chymase on IL-18 levels, 1-300 nM of the recombinant chymase were incubated with saliva samples at 37° C. for 1 hr. The results, which are summarized in FIG. 5, show that the chymase-induced IL-18 levels were dose dependent. The EC₅₀ of the recombinant chymase was estimated at 60.4±15.2 nM (n=3).

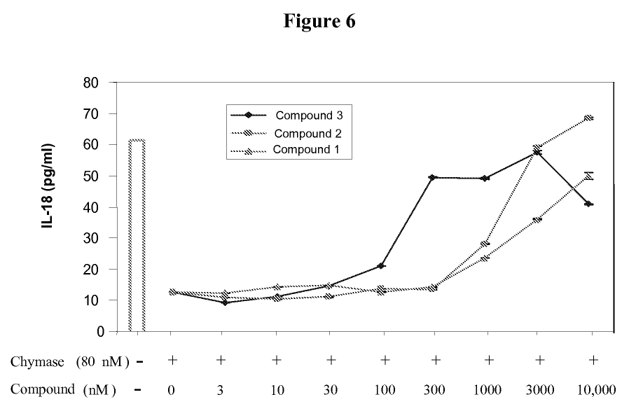

The dose effects of the chymase inhibitors on IL-18 levels were also examined. Compound Nos. 1, 2 and 3 in 0, 3, 10, 30, 100, 300, 1000, 3000, and 10,000 nM were incubated with 80 nM of chymase and IL-18 at 37° C. for 1 hr. The IL-18 levels were measured using ELISA as described above. The results in FIG. 6 show that all three chymase inhibitors reversed the chymase-induced IL-18 reduction. Similarly, the effects were dose dependant. The IC₅₀ values for COMPOUND Nos. 1, 2 and 3 were estimated at 125.1±43.2 nM, 1456.0±562.7 nM, and 3602.7±928.1 nM, respectively (n=3).

### Example 4

### Specificity of Serine Protease and Inhibitors

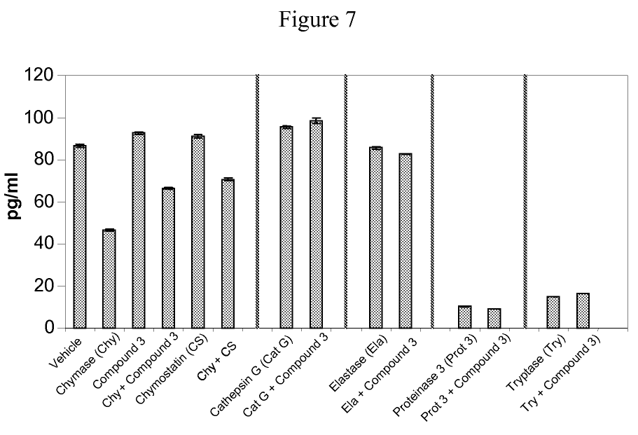

Next, the effects of various serine proteases and inhibitors on IL-18 levels were examined. The saliva samples were incubated with vehicle (DMSO/PBS), chymase (2 μg/ml), COMPOUND No. 3 (1 μM), chymase (2 μg/ml)+COMPOUND No. 3 (1 μM), chymostatin (3 μg/ml), chymase (2 μg/ml)+chymostatin (3 μg/ml), cathepsin G (2 μg/ml), cathepsin G (2 μg/ml)+COMPOUND No. 3 (1 μM), elastase (0.5 μg/ml), elastase (0.5 μg/ml)+COMPOUND No. 3 (1 μM), proteinase 3 (2 μg/ml), proteinase 3 (2 μg/ml)+COMPOUND No. 3 (1 μM), tryptase (1 μg/ml), or tryptase (1 μg/ml)+COMPOUND 3 (1 μM) at 37° C. for 1 h. IL-18 levels were measured by ELISA as described above and summarized in FIG. 7. Similar to the results shown above, chymase reduced the IL-18 levels and COMPOUND No. 3 reversed this reduction. Additionally, chymostatin, a nonspecific chymase inhibitor, also reversed the reduction induced by chymase. In contrast, cathepsin G and elastase had no effect on IL-18 levels. Proteinase 3 and tryptase reduced IL-18 levels significantly. However, the reduction of IL-18 by these two enzymes was not reversed by COMPOUND No. 3. These results indicate that the effect of Compound No. 3 was specific to the chymase.

### Example 5

### Dose Response Studies of COMPOUND NO. 1

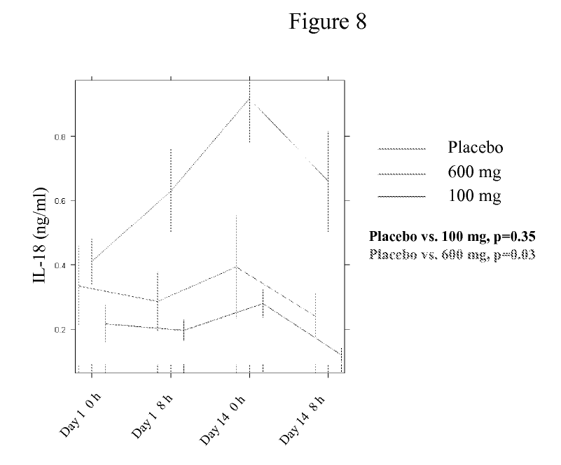

To examine the dose effects of COMPOUND NO. 1 on the IL-18 levels, subjects were treated with 0, 100, 600 mg of COMPOUND NO. 1 for 14 days and the saliva samples were collected. IL-18 levels in the samples were measured by ELISA as described above. A significant increase (p<0.03) of the IL-18 levels was detected in the subjects treated with COMPOUND NO. 1 at 600 mg as compared to the placebo group (FIG. 8). The IL-18 levels were similar in the placebo group and the subjects treated with COMPOUND NO. 1 at 100 mg.

While the foregoing specification teaches the principles of the present invention, with examples provided for the purpose of illustration, it will be understood that the practice of the invention encompasses all of the usual variations, adaptations and modifications as come within the scope of the following claims and their equivalents.

### REFERENCES

Bombardieri M, Barone V, Pittoni V, et al. 2004. Increased circulating levels and salivary gland expression of interleukin-18 in patients with Sjogren's syndrome: relationship with autoantibody production and lymphoid organization of the periductal inflammatory infiltrate. Arthritis Res. Ther 6:447-456.
Caughey G. 2007. Mast cell tryptases and chymases in inflammation and host defense. Immunol Review 217:141-154.
M. Cockett et al., 2000, Current Opinion in Biotechnology, 11:602-609
Kervinen J. Abad M, Crysler C, Kolpak M, Mahan A, Masucci J, Bayoumy S, Cummings M, Yao X, Olson, M, Garavilla, L, Kuo L, Deckman I, Spurlino J. 2008. Structural basis for elastolytic substrate specificity in rodent chymases. J Biological Chemistry 283:427-436.
Nakanishi K, Tomohiro Y, Tshusui H, Okamura H. 2001. Interleukin-18 is a unique cytokine that stimulates both Th1 and Th2 responses depending on its cytokine milieu. Cytokine & Growth Factor Rev 12:53-72.
Okamura H, Nagata K, Komatsu T, et al. 1995a. A novel costimulatory factor for gamma interferon induction found in the liver of mice causes endotoxic shock. Infect Immunol 63:3966-72.
Okamura H, Tsutsui H, Komatsu T, et al. 1995b. Cloning of a new cytokine that induces IFN-r production by T cells. Nature 378: 88-91.
Omoto Y, Tokime K, Yamanaka k, et al. 2006. Human mast cell chymase cleaves pro-IL-18 and generates a novel and biologically active IL-18 fragment. J Immunol 177:8315-8319.

1. A biomarker predictive of a response of cells to treatment with a drug that inhibits chymase activity.

2. The biomarker of claim 1, wherein the biomarker is IL-18.

3. A method of predicting whether a drug is capable of inhibiting chymase activity in a cell, comprising: a) obtaining a first sample of cells prior to administration of said drug, b) obtaining a second sample of cells after administration of said drug; c) determining the immunoreactivity of IL-18 in said first and second samples; d) comparing the immunoreactivity of IL-18 in said samples; and e) correlating any change in the immunoreactivity of IL-18 to said drugs' ability or inability to inhibit chymase activity in said subject.

4. The method of claim 3, wherein said subject is human.

5. The method of claim 3, wherein said first and second samples are collected from saliva.

6. A method of screening for a candidate drug capable of inhibiting the activity of chymase, comprising: a) contacting a test drug with a sample of cells; and b) selecting as a candidate those drugs that increase IL-18 in said sample of cells.
